(19) United States
(12) Patent Application Publication
(10) Pub. No.: US 2014/0088359 A1
(43) Pub. Date: Mar. 27, 2014

(54) Nasogastric Scope

(71) Applicant: Roland A. QUAYE, Fredericksburg, VA (US)

(72) Inventor: Roland A. QUAYE, Fredericksburg, VA (US)

(21) Appl. No.: 13/899,778

(22) Filed: May 22, 2013

Related U.S. Application Data
(60) Provisional application No. 61/704,617, filed on Sep. 24, 2012.

(51) Int. Cl. A61B 1/233; A61B 1/00

(52) U.S. Cl. CPC A61B 1/233; A61B 1/00114
USPC 600/110

(57) ABSTRACT

A nasogastric scope that can be inserted into an individual without sedating said individual. The nasogastric scope has a nasogastric tube with an attached viewing device. The nasogastric tube is used to feed a patient or empty the contents of a patient's stomach, while the viewing device has a camera which allows a user to observe the positioning of the nasogastric tube within a patient, as well as observe any obstructions or irregularities in a patient's stomach. An articulation mechanism can be used to change the direction in which the camera is oriented. Images captured by the camera are transmitted to a monitoring device where they can be seen by the user on a display screen and stored on a chipset. The monitoring device also provides a control panel that allows the user to input commands in order to control the camera, articulation mechanism, and chipset.

The current application claims a priority to the U.S. Provisional Patent application Ser. No. 61/704,617 filed on Sep. 24, 2012.

### FIELD OF THE INVENTION

The present invention relates generally to a medical device. More specifically, the present invention provides the benefits of both a nasogastric tube and an endoscope in a single medical device.

### BACKGROUND OF THE INVENTION

Nasogastric tubes are used on a daily basis in hospitals as feeding tubes and to empty the contents of a patient's stomach. A nasogastric tube is inserted through a patient's nose or mouth, down into their stomach. While inserting a nasogastric tube, it is important to make sure that the nasogastric tube enters the patient's stomach and not their lungs. Therefore, once a nasogastric tube has been inserted it is common practice for an x-ray to be taken to ensure the proper placement of the nasogastric tube. This not only takes time and money but exposes the patient to radiation.

Nasogastric tubes are also used in upper gastrointestinal bleeding, bowel obstruction and in comatose patients, among other uses. However, in order to view the source of bleeding or the obstruction an upper endoscope must be used. This requires that the patient be put under anesthesia or heavy sedation. It is preferable to avoid inducing a patient as not all people may have the same reaction to sedatives or anesthesia and the improper use of sedatives could be lethal.

Therefore it is the object of the present invention to provide a nasogastric scope that can be inserted without the use of heavy sedatives. The nasogastric scope functions as a standard nasogastric tube with the inclusion of an integrated camera. This also eliminates the need for a post insertion x-ray, as the user can see where the nasogastric scope is being inserted. In turn, time and money are saved by eliminating the previously needed steps to check for the proper insertion of the tube. Additionally, the patient does not need to be exposed to any unnecessary radiation.

### BRIEF DESCRIPTION OF THE DRAWINGS

FIG. 1 is a perspective view of the nasogastric scope.

FIG. 2 is a perspective view of the interior tube end of the nasogastric tube

FIG. 3 is a perspective view of the camera being pivoted on the interior tube end of the nasogastric tube.

FIG. 4 is a perspective view of the exterior tube end of the nasogastric tube.

FIG. 5 is a lateral cross sectional view of the nasogastric tube.

FIG. 6 is a longitudinal cross sectional view of the nasogastric tube.

FIG. 7 is a perspective view of the monitoring device.

FIG. 8 is a schematic of the electrical connections of both the monitoring device and the viewing device.

FIG. 9 is a schematic of the electronic connections of both the monitoring device and the viewing device.

FIG. 10 is a schematic of the electronic connections of both the monitoring device and the viewing device with the monitoring device having a touch screen.

FIG. 11 is a perspective view of the hand-held device.

FIG. 12 is a schematic of the electrical connections of the hand-held device, monitoring device and viewing device.

FIG. 13 is a schematic of the electronic connections of the hand-held device, monitoring device and viewing device.

FIG. 14 is a schematic of the electrical connections of the monitoring device and the viewing device, while the monitoring device is wirelessly communicating with the viewing device.

FIG. 15 is a schematic of the electronic connections of the monitoring device and the viewing device, while the monitoring device is wirelessly communicating with the viewing device.

### DETAIL DESCRIPTIONS OF THE INVENTION

All illustrations of the drawings are for the purpose of describing selected versions of the present invention and are not intended to limit the scope of the present invention.

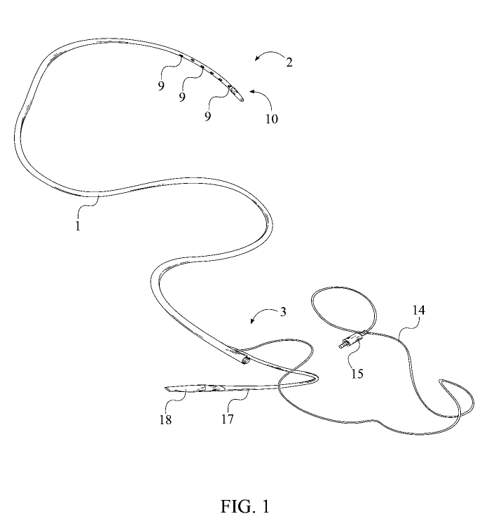

The present invention is a nasogastric scope that can be used without putting a patient under heavy sedatives or anesthesia and does not require additional means to check for the correct positioning of the tube. In general, the nasogastric scope is used for upper gastrointestinal bleeding, bowel obstruction, stomach pumping and as a feeding tube, however, it is possible that it be used for any number of other purposes. The nasogastric scope comprises a nasogastric tube 1, a vent tube 17, a viewing device 10, and a monitoring device 19. The nasogastric tube 1 is inserted into the stomach of an individual and allows a user to feed the individual or remove waste from the stomach of the individual. In reference to FIG. 1, the viewing device 10 is attached to one end of the nasogastric tube 1 and captures images and video, which are transmitted to the monitoring device 19 where they can then be viewed by the user. In this way, the user can ensure that the nasogastric tube 1 is properly positioned within the individual's stomach, as well as view any potential obstructions, bleeding, etc. The nasogastric scope can be designed such that the nasogastric tube 1 is disposable or such that the nasogastric tube 1 can be sterilized and re-used.

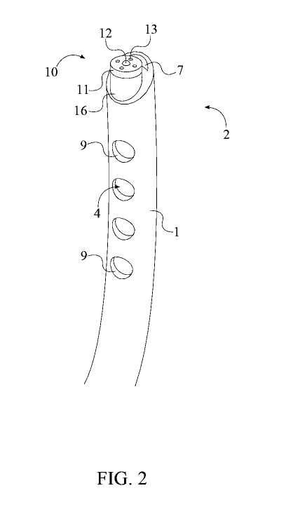

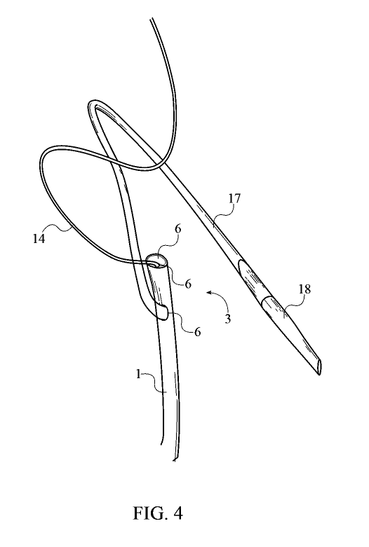

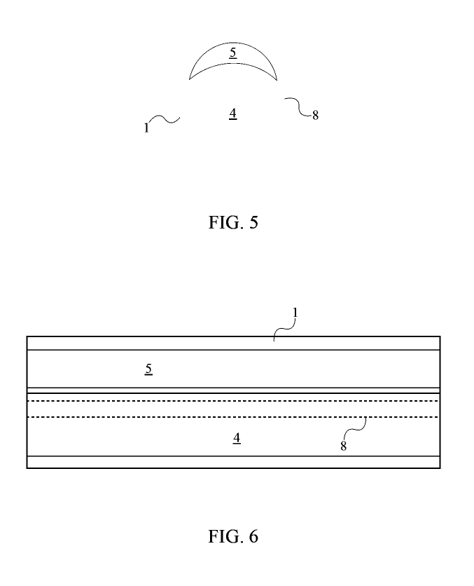

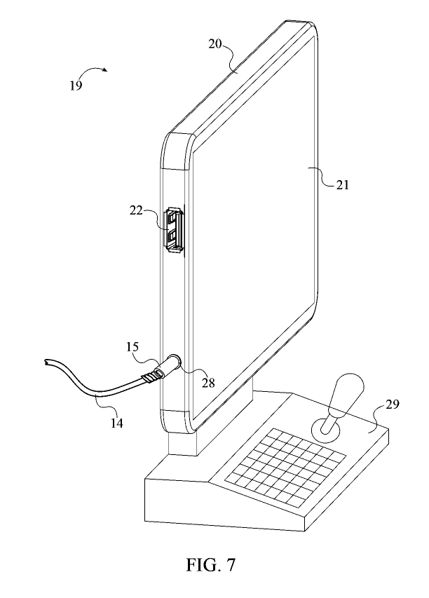

In reference to FIG. 2-6, in the preferred embodiment of the present invention, the nasogastric tube 1 is made of a silicon or rubber material; however, it is possible that any number of other materials be used given preference to flexible materials. The nasogastric tube 1 can be designed in different lengths and diameters in order to allow use with both children and adults. The nasogastric tube 1 comprises an interior tube end 2, an exterior tube end 3, a suction lumen 4, a vent lumen 5, a cable lumen 8, and a plurality of suction holes 9. The interior tube end 2 and the exterior tube end 3 are positioned opposite of each other along the nasogastric tube 1, with the interior tube end 2 being positioned within the human body. In the preferred embodiment of the present invention, the interior tube end 2 is pointed in order to allow for easier insertion of the nasogastric tube 1 into an individual. A stomach pump, feeding pump or similar device can be attached along the exterior tube end 3, which remains outside of the individual.

In reference to FIG. 2-6, the suction lumen 4 is the channel in the nasogastric tube 1 through which waste is removed from an individual's stomach or through which food is inserted into an individual's stomach. The suction lumen 4 is positioned both along and within the nasogastric tube 1, and comprises an external open end 6. The external open end 6 is positioned through the exterior tube end 3 and allows contents to enter or be removed from the suction lumen 4 using a food pump, stomach pump, or similar device that is attached to the exterior tube end 3. Food is inserted or waste is removed from an individual's stomach via the plurality of suction holes 9, which are positioned adjacent to the interior tube end 2. The plurality of suction holes 9 laterally traverses through the nasogastric tube 1 into the suction lumen 4, allowing contents from the individual's stomach to enter the nasogastric tube 1 and vice versa.

In reference to FIG. 2-6, the vent lumen 5 is the channel in the nasogastric tube 1 which assists the suction channel in the removal of contents from an individual's stomach. The vent lumen 5 is positioned both along and within the nasogastric tube 1, and comprises an internal open end 7 and an external open end 6. The internal open end 7 is positioned through the interior tube end 2, while the external open end 6 of the vent lumen 5 intersects through the nasogastric tube 1 adjacent to the exterior tube end 3. The internal open end 7 and the external open end 6 of the vent lumen 5 allow air to enter or exit an individual's stomach through the vent lumen 5 while the nasogastric tube 1 is being used to suction. This flow of air prevents the plurality of suction holes 9 from suctioning to the walls of an individual's stomach and damaging the lining of the stomach. The vent tube 17 is adjacently attached to the external open end 6 of the vent lumen 5, and comprises a ventilation valve 18. The ventilation valve 18 is positioned opposite of the external open end 6 of the vent lumen 5 along the vent tube 17. The vent tube 17 serves as an extension of the vent lumen 5, while the ventilation valve 18 prevents contents accidentally suctioned through the vent lumen 5 from exiting.

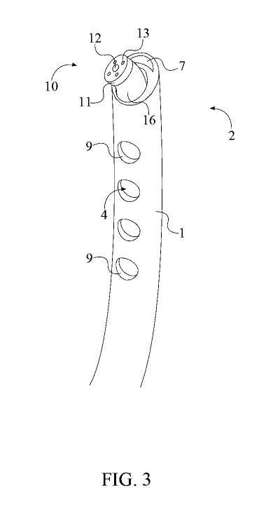

In reference to FIG. 2-3, the nasogastric scope has a viewing device 10, which allows a user to capture images and/or video when the nasogastric scope is inserted into an individual. The viewing device 10 comprises a camera 11, a camera cable 14, and an articulation mechanism 16. Images are captured by the camera 11 and transmitted to the monitoring device 19 through the camera cable 14. The camera 11 comprises a camera lens 12 and an at least one light 13. The camera lens 12 allows the camera 11 to capture images, while the at least one light 13 is positioned around the camera lens 12 and provides adequate illumination of the stomach such that the camera lens 12 can capture said images. In the preferred embodiment of the present invention, the at least one light 13 is a light emitting diode, however, any other type of light source can be used. Additionally, the camera lens 12 can be any type of lens, such as a wide angle lens or pin-hole lens. Both the camera lens 12 and the at least one light 13 are sealed in order to prevent damage to the camera 11 due to contents in the stomach.

In reference to FIG. 2-3, the camera 11 is pivotally attached to the interior tube end 2 by the articulation mechanism 16. The camera 11 may be attached by a threaded connection, snap fit or by any other ample means of connection. It is possible for the articulation mechanism 16 to be made detachable from the nasogastric tube 1 or to be permanently affixed. The articulation mechanism 16 allows the user to adjust the direction in which the camera lens 12 is facing by rotating or otherwise altering the position of the camera 11. This allows a user to more readily view the contents of an individual's stomach. The camera cable 14 is attached to both the camera 11 and the articulation mechanism 16, and is positioned within the cable lumen 8. The cable lumen 8 separates the camera cable 14 from the suction lumen 4 and the vent lumen 5, and prevents the camera cable 14 from getting wet or otherwise damages. The cable lumen 8 is positioned both along and within the nasogastric tube 1 and comprises an external open end 6. The external open end 6 of the cable lumen 8 is positioned though the exterior tube end 3 and allows the camera cable 14 to exit the nasogastric tube 1. The camera cable 14 comprises a cable adapter 15. The cable adapter 15 is positioned opposite of the camera 11 and the articulation mechanism 16 along the camera cable 14, such that it is positioned externally of the nasogastric tube 1.

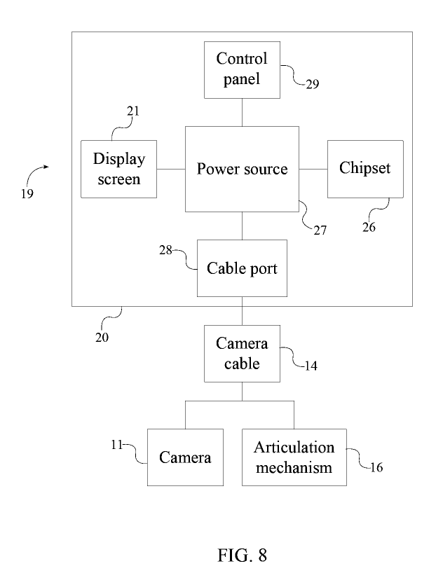

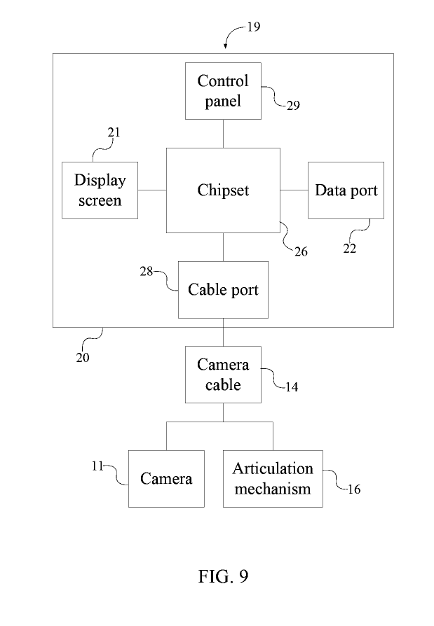

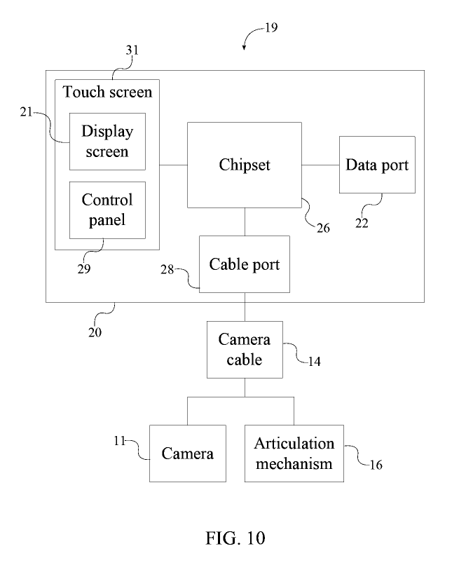

In reference to FIG. 7, the camera cable 14 attaches the viewing device 10 to the monitoring device 19, such that images captured by the camera 11 can be viewed by the user. The monitoring device 19 comprises a housing 20, a display screen 21, a chipset 26, a power source 27, an at least one data port 22, a cable port 28, and a control panel 29. The chipset 26 is a series of integrated circuits and is electronically connected to the display screen 21, the at least one data port 22, the cable port 28, and the control panel 29, as shown in FIG. 9. The chipset 26 manages and instructs the components to which it is connected and may include a processor or similar device. The chipset 26 also has an internal storage means, which allows images captured by the camera 11 to be saved. Power is supplied to the chipset 26 by the power source 27. The power source 27 provides electrical power to the components of the monitoring device 19, and is electrically connected to the display screen 21, chipset 26, cable port 28, and control panel 29, as shown in FIG. 8. In the one embodiment of the present invention, the power source 27 provides alternating current, which is supplied from a standard power outlet to the power source 27 through a power cable. In another embodiment of the present invention, the power source 27 provides direct current. Direct current can be supplied by a battery, which allows the monitoring device 19 to be used away from a fixed source of power. Both the chipset 26 and the power source 27 are positioned within the housing 20. The housing 20 protects the chipset 26 and the power source 27 from being damaged.

In reference to FIG. 7, the housing 20 also provides a casing for mounting the display screen 21, at least one data port 22, cable port 28 and control panel 29. The display screen 21 is mounted into one side of the housing 20, while the at least one data port 22 and cable port 28 traverse into one or more sides of the housing 20 adjacent to the display screen 21. The cable port 28 is both electronically and electrically attached to the camera cable 14 by the cable adapter 15. The camera cable 14 is also electronically and electrically connected to both the camera 11 and the articulation mechanism 16. In this way, the power source 27 is able to provide power to the viewing device 10, while the chipset 26 can provide instructions to the camera 11 and articulation mechanism 16. Images captured by the camera 11 can be transmitted to the chipset 26 through the camera cable 14 and cable port 28. These images can then be stored on the chipset 26 or transmitted to the display screen 21 where they can be viewed by the user.

Functions of the camera 11 and articulation mechanism 16 can also be managed by the control panel 29 through the chipset 26. The control panel 29 allows the user to adjust the focus of the camera lens 12, adjust the brightness level of the at least one light 13, control movements of the articulation mechanism 16, etc. The control panel 29 is also used to operate the chipset 26 in order to save images, access and manipulate stored images, transfer images, etc. In one embodiment of the present invention, the control panel 29 is positioned on the housing 20 adjacent to the display screen 21. In another embodiment of the present invention, a touch screen 31 is both the display screen 21 and the control panel 29, allowing the monitoring device 19 to be more portable. Images stored on the chipset 26 can be transferred to an external storage device through the at least one data port 22. The at least one data port 22 can be any type of port such as a universal serial bus port, high-definition multimedia interface port, digital visual interface port, or video graphics array port.

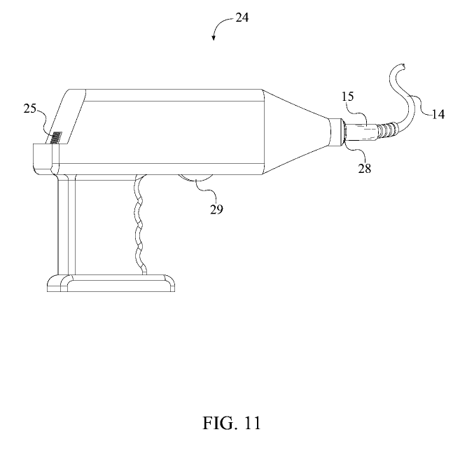

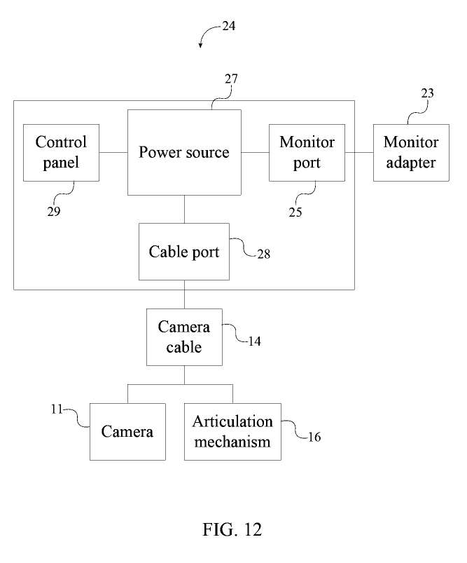

In reference to FIG. 11, the nasogastric scope may also comprise a hand-held device 24 to which the monitoring device 19 may be attached. The hand-held device 24 comprises a power source 27, a cable port 28, and a monitor port 25. The monitor port 25 and the cable port 28 of the hand-held device 24 are electronically connected to each other and are positioned opposite of each other along the hand-held device 24, such that the viewing device 10 and monitoring device 19 are attached to opposite ends of the hand-held device 24. The camera cable 14 is both electronically and electrically attached to the cable port 28 of the hand-held device 24 by the cable adapter 15, while the monitoring device 19 further comprises a monitor adapter 23, which is both electronically and electrically attached to the monitor port 25. The power source 27 of the hand-held device 24 provides direct current, is positioned within the hand-held device 24, and is electrically connected to both the monitor port 25 and the cable port 28 of the hand-held device 24. In this way, the power source 27 of the hand-held device 24 supplies power to both the monitoring device 19 and the viewing device 10 through the monitor port 25 and cable port 28 of the hand-held device 24, respectively, as shown in FIG. 12.

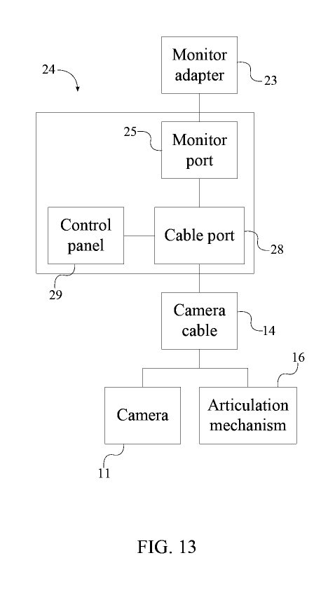

In reference to FIG. 13, the monitor port 25 and the cable port 28 of the hand-held device 24 are also electronically connected to each other. This allows the user to control the camera 11 and articulation mechanism 16 by the chipset 26 in the monitoring device 19. It is also possible for the hand-held device 24 to further comprise a control panel 29. The control panel 29 of the hand-held device 24 is positioned in between the monitor port 25 and the cable port 28 of the hand-held device 24, such that it can be easily accessed by a user's hand while holding the hand-held device 24. The power source 27 is electrically connected to the control panel 29 of the hand-held device 24 and provides direct current to the control panel 29 of the hand-held device 24. The control panel 29 of the hand-held device 24 is also electronically connected to the monitor port 25, which allows input commands to be processed by the chipset 26 of the monitoring device 19. The control panel 29 of the hand-held device 24 and the control panel 29 of the monitoring device 19 may provide redundant controls or controls that supplement one another. For example, the control panel 29 of the monitoring device 19 may only be used to operate the display screen 21 and memory functions of the chipset 26, while the control panel 29 of the hand-held device 24 is used to operate the camera 11 and the articulation mechanism 16.

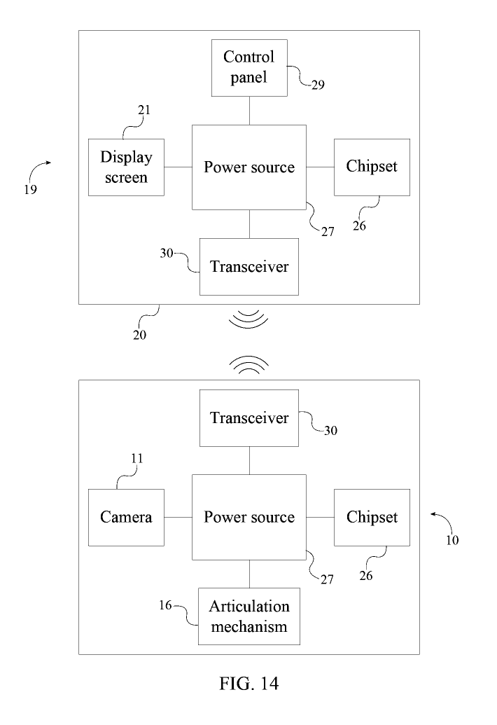

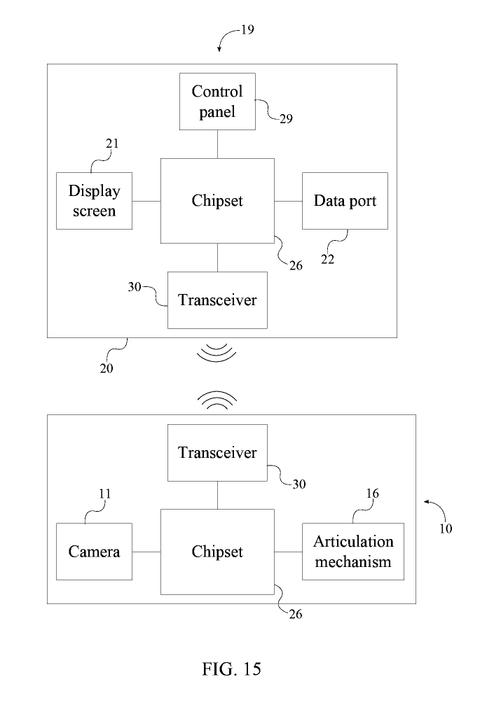

In reference to FIG. 14-15, in an alternative embodiment of the present invention, the nasogastric scope is designed such that it is wireless. In this embodiment, the nasogastric tube 1 does not comprise a cable lumen 8 and the camera cable 14 of the viewing device 10 is replaced by a transceiver 30, a chipset 26, and a power source 27. Additionally, the monitoring device 19 further comprises a transceiver 30. The transceiver 30 of the monitoring device 19 is electrically connected to the power source 27 of the monitoring device 19 and electronically connected to the chipset 26 of the monitoring device 19. Similarly, the transceiver 30 of the viewing device 10 is electrically connected to the power source 27 of the viewing device 10 and electronically connected to the chipset 26 of the viewing device 10. Both the transceiver 30 of the viewing device 10 and the transceiver 30 of the monitoring device 19 are communicably coupled, such that data may be transmitted between one another. In this way, images captured by the camera 11 can be wirelessly transferred to the monitoring device 19. The chipset 26 of the viewing device 10 is also electronically connected to the camera 11 and the articulation mechanism 16. This allows commands that are sent from the monitoring device 19 and received by the transceiver 30 of the viewing device 10 to be processed and used to operate the camera 11 and/or articulation mechanism 16. The power source 27 of the viewing device 10 is also electrically connected to the camera 11, the articulation mechanism 16, and the chipset 26 of the viewing device 10, providing a source of direct current to the components of the viewing device 10.

Although the invention has been explained in relation to its preferred embodiment, it is to be understood that many other possible modifications and variations can be made without departing from the spirit and scope of the invention as hereinafter claimed.

1. A nasogastric scope comprises:
a nasogastric tube;
a viewing device;
a vent tube;
a monitoring device;
the nasogastric tube comprises an interior tube end, an exterior tube end, a suction lumen, a vent lumen, a cable lumen, and a plurality of suction holes;
the viewing device comprises a camera, a camera cable and an articulation mechanism;
the vent tube comprises a ventilation valve;
the monitoring device comprises a housing, a display screen, a chipset, a power source, an at least one data port, a cable port, and a control panel;
the suction lumen and the vent lumen each comprise an external open end;
the vent lumen further comprises an internal open end;
the cable lumen comprises an external open end;
the camera comprises a camera lens and an at least one light;
the camera cable comprises a cable adapter;
the interior tube end and the exterior tube end being positioned opposite of each other along the nasogastric tube; and
the suction lumen, the vent lumen, and the cable lumen being positioned both along and within the nasogastric tube.

2. The nasogastric scope as claimed in claim 1 comprises:
the plurality of suction holes laterally traversing through the nasogastric tube into the suction lumen;
the plurality of suction holes being positioned adjacent to the interior tube end;
the external open end of both the suction lumen and the cable lumen being positioned through the exterior tube end;
the internal open end being positioned through the interior tube end;
the external open end of the vent lumen intersecting through the nasogastric tube adjacent to the exterior tube end;
the vent tube being adjacently attached to the external open end of the vent lumen; and
the ventilation valve being positioned opposite of the external open end of the vent lumen along the vent tube.

3. The nasogastric scope as claimed in claim 1 comprises:
the camera being pivotally attached to the interior tube end by the articulation mechanism;
the camera cable being positioned within the cable lumen;
the cable adapter being positioned opposite of the camera and articulation mechanism along the camera cable;
the at least one light being positioned around the camera lens; and
the camera and the articulation mechanism being both electronically and electrically connected to the camera cable.

4. The nasogastric scope as claimed in claim 1 comprises:
the display screen being mounted into the housing;
the chipset and the power source being positioned within the housing; and
the at least one data port and the cable port traversing into the housing.

5. The nasogastric scope as claimed in claim 4 comprises:
the control panel being positioned on the housing adjacent to the display screen.

6. The nasogastric scope as claimed in claim 4 comprises:
a touch screen being both the display screen and the control panel.

7. The nasogastric scope as claimed in claim 1 comprises:
the display screen, the at least one data port, the cable port, and the control panel being electronically connected to the chipset;
the display screen, the chipset, the cable port, and the control panel being electrically connected to the power source;
the camera cable being both electronically and electrically attached to the cable port; and
the cable adapter being attached to the cable port.

8. The nasogastric scope as claimed in claim 1 comprises:
the nasogastric scope further comprises a hand-held device;
the hand-held device comprises a power source, a cable port, and a monitor port;
the monitoring device further comprises a monitor adapter;
the power source being positioned within the hand-held device; and
the monitor port and the cable port of the hand-held device being positioned opposite of each other along the hand-held device.

9. The nasogastric scope as claimed in claim 8 comprises:
the cable port of the hand-held device being electronically connected to the monitor port;
the monitor port and the cable port of the hand-held device being electrically connected to the power source of the hand-held device;
the camera cable being both electronically and electrically attached to the cable port of the hand-held device;
the cable adapter being attached to the cable port of the hand-held device; and
the monitor adapter being both electronically and electrically attached to the monitor port.

10. The nasogastric scope as claimed in claim 8 comprises:
the hand-held device further comprises a control panel;
the control panel of the hand-held device being positioned in between the monitor port and the cable port of the hand-held device;
the control panel of the hand-held device being electronically connected to the monitor port; and
the control panel of the hand-held device being electrically connected to the power source of the hand-held device.

11. A nasogastric scope comprises:
a nasogastric tube;
a viewing device;
a vent tube;
a monitoring device;
the nasogastric tube comprises an interior tube end, an exterior tube end, a suction lumen, a vent lumen, and a plurality of suction holes;
the viewing device comprises a camera, an articulation mechanism, a transceiver, a chipset, and a power source;
the vent tube comprises a ventilation valve;
the monitoring device comprises a housing, a display screen, a chipset, a power source, an at least one data port, a control panel, and a transceiver;
the suction lumen and the vent lumen each comprise an external open end;
the vent lumen further comprises an internal open end;
the cable lumen comprises an external open end;
the camera comprises a camera lens and an at least one light;
the interior tube end and the exterior tube end being positioned opposite of each other along the nasogastric tube; and
the suction lumen and the vent lumen being positioned both along and within the nasogastric tube.

12. The nasogastric scope as claimed in claim 11 comprises:
the plurality of suction holes laterally traversing through the nasogastric tube into the suction lumen;
the plurality of suction holes being positioned adjacent to the interior tube end;
the external open end of both the suction lumen and the cable lumen being positioned through the exterior tube end;
the internal open end being positioned through the interior tube end;
the external open end of the vent lumen intersecting through the nasogastric tube adjacent to the exterior tube end;
the vent tube being adjacently attached to the external open end of the vent lumen; and
the ventilation valve being positioned opposite of the external open end of the vent lumen along the vent tube.

13. The nasogastric scope as claimed in claim 11 comprises:
the camera being pivotally attached to the interior tube end by the articulation mechanism;
the at least one light being positioned around the camera lens;
the camera, the articulation mechanism, and the transceiver of the viewing device being electronically connected to the chipset of the viewing device; and
the camera, the articulation mechanism, the transceiver of the viewing device, and the chipset of the viewing device being electrically connected to the power source of the viewing device.

14. The nasogastric scope as claimed in claim 11 comprises:
the display screen being mounted into the housing;
the chipset, the transceiver of the monitoring device, and the power source of the monitoring device being positioned within the housing; and
the at least one data port traversing into the housing.

15. The nasogastric scope as claimed in claim 14 comprises:
the control panel being positioned on the housing adjacent to the display screen.

16. The nasogastric scope as claimed in claim 14 comprises:
a touch screen being both the display screen and the control panel.

17. The nasogastric scope as claimed in claim 11 comprises:
the display screen, the at least one data port, the control panel, and the transceiver of the monitoring device being electronically connected to the chipset of the monitoring device;
the display screen, the chipset of the monitoring device, the control panel, and the transceiver of the monitoring device being electrically connected to the power source of the monitoring device; and
the transceiver of the monitoring device being communicably coupled to the transceiver of the viewing device.

18. The nasogastric scope as claimed in claim 11 comprises:
the nasogastric scope further comprises a hand-held device;
the hand-held device comprises a power source and a monitor port;
the monitoring device further comprises a monitor adapter; and
the power source of the hand-held device being positioned within the hand-held device.

19. The nasogastric scope as claimed in claim 18 comprises:
the monitor port being electrically connected to the power source of the hand-held device; and
the monitor adapter being both electronically and electrically attached to the monitor port.

20. The nasogastric scope as claimed in claim 18 comprises:
the hand-held device further comprises a control panel;
the control panel being positioned adjacent to the monitor port;
the control panel of the hand-held device being electronically connected to the monitor port; and
the control panel of the hand-held device being electrically connected to the power source of the hand-held device.
